(19) United States
(12) Patent Application Publication
(10) Pub. No.: US 2024/0238272 A1
(43) Pub. Date: Jul. 18, 2024

(54) TREATMENT OF ANTIPHOSPHOLIPID SYNDROME USING S-HYDROXYCHLOROQUINE

(71) Applicant: Genovate Biotechnology Co. Ltd., Hsinchu (TW)

(72) Inventors: Jen Chen, Hsinchu (TW); Chia-Chen Chu, Hsinchu (TW)

(21) Appl. No.: 18/561,712

(22) PCT Filed: Jun. 1, 2022
(86) PCT No.: PCT/US2022/031806

§ 371 Date: Nov. 16, 2023

Related U.S. Application Data
(60) Provisional application No. 63/197,033, filed on Jun. 4, 2021.

(51) Int. Cl. A61K 31/4706; A61P 7/02

(52) U.S. Cl. CPC A61K 31/4706; A61P 7/02

(57) ABSTRACT

A method for treating antiphospholipid syndrome by administering to a patient a pharmaceutical composition that contains S-(+)-hydroxychloroquine and a pharmaceutically acceptable excipient. The pharmaceutical composition is substantially free of R-(−)-hydroxychloroquine.

### BACKGROUND

Antiphospholipid syndrome (“APS”), an autoimmune disorder associated with thrombosis and pregnancy losses, can cause life-threatening blood clots in lungs and brain. APS is a leading cause of strokes in people under 50 years old. In pregnant women, it often results in miscarriage and still birth.

According to a report, APS affects 0.3-1% of the population. See McDonnell et al., Blood Review 39, 1-14 (2020). There is currently no cure. Warfarin, a long-term anticoagulant medication, is a standard treatment for APS-associated thrombosis to reduce risk of blood clots. However, it carries significant risk of bleeding complications. See Rand et al., Blood 112, 1687-95 (2008).

For additional references on APS, see Agar et al., Blood 116, 1336-43 (2010); Rand et al., Blood 115, 2292-99 (2010); Rand et al., Lupus 17, 922-30 (2008); and Conti et al., Clin Exp Immunol 132, 509-16 (2003).

Hydroxychloroquine (“HCQ”), an antimalaria compound, was suggested to play a role in reducing the extend of thrombosis in an animal model of injury-induced thrombosis and reversing antiphospholipid (“aPL”) antibody-induced platelet activation. See Rand et al. (2008). Its efficacy and safety are yet to be determined in large scale clinical studies.

HCQ has two optical isomers, i.e., the (R)-(−)-isomer (“R-HCQ”) and the (S)-(+) isomer (“S-HCQ”). A racemic mixture, containing R-HCQ and S-HCQ at 50:50, were used for all studies mentioned above.

Long-term and high-dose administration of HCQ can cause blurred vision and in some cases can damage the retina, cornea, or macula and lead to vision impairments in some patients due to its accumulation in ocular tissues.

HCQ is also known to be cardiotoxic. It can cause interventricular conduction delay, Q wave to T wave interval prolongation, Torsades de pointes, ventricular arrhythmia, hypokalemia, and hypotension. See U.S. patent application Ser. No. 17/176,679.

There is a need to develop a method for effectively treating APS in a safe manner.

### SUMMARY

To meet the above need, a method is provided for treating APS with a pharmaceutical composition that contains S-(+)-hydroxychloroquine (“S-HCQ”) and a pharmaceutically acceptable excipient.

Accordingly, this invention relates to a method of treating APS includes the steps of: (i) identifying a subject suffering from APS and (ii) administering to the subject an effective amount of a pharmaceutical composition containing S-HCQ and a pharmaceutically acceptable excipient, thereby treating APS. The pharmaceutical composition is substantially free of (R)-(−)-hydroxychloroquine (“R-HCQ”).

The method of this invention is suitable for treating APS of all types, e.g., primary APS, secondary APS, and catastrophic APS.

The pharmaceutical composition is administered in any form including granules, a tablet, a capsule, a pill, a powder, a solution, a suspension, or a syrup. Preferably, it is administered to a patient at a dose of 100 mg to 800 mg (e.g., 120 mg to 600 mg, 150 mg to 500 mg, and 180 mg to 450 mg) of S-HCQ per day.

S-HCQ refers to the compound itself and a pharmaceutically acceptable salt thereof. Examples of its salt are a hydrochloride salt, a sulfate salt, and a phosphate salt.

The details of several embodiments of the present invention are set forth in both the description and the drawing below. Other features, objects, and advantages of the invention will be apparent from the description and also from the appended claims. Finally, all publications and patent documents cited herein are incorporated by reference in their entirety.

### BRIEF DESCRIPTION OF THE DRAWINGS

The description below refers to the accompanying drawing.

FIG. 1 includes pictures obtained from a molecular docking study, showing a HCQ (S-HCQ or R-HCQ) molecular binding to β2-glycoprotein I that has a structure in the form of a-helix including four joints.

### DETAILED DESCRIPTION

As summarized above, a method is provided for treating APS by administering to an APS patient a pharmaceutical composition that contains high-purity S-HCQ and a pharmaceutically acceptable excipient, the pharmaceutical composition being substantially free of R-HCQ.

The purity of S-HCQ is measured by its enantiomeric excess, defined as the molar percentage difference between S-HCQ and R-HCQ, in which the total molar percentages of S-HCQ and R-HCQ is 100%. For example, the purity of S-HCQ in an enantiomeric excess of 99% contains 99.5% by mole of S-HCQ and 0.5% by mole of R-HCQ. The pharmaceutical composition is deemed substantially free of R-HCQ when it contains S-HCQ in an enantiomeric excess of 99% or greater (e.g., 99.2% or greater, 99.5% or greater, and 99.8% or greater).

S-HCQ preparations having such a high enantiomeric excess is described in U.S. patent application Ser. No. 17/176,679 and U.S. Pat. No. 5,314,894.

The S-HCQ in the pharmaceutical composition is either a free base or a pharmaceutically acceptable salt. The pharmaceutically acceptable salt can be, but is not limited to, a sulfate salt, a phosphate salt, and a hydrochloride salt. Preferably, it is a sulfate salt.

The pharmaceutical composition contains by weight S-HCQ in the range of 5% to 95%, e.g., 30% to 80%, 40% to 70%, 40% to 55%, and 60% to 70%.

The pharmaceutical composition is substantially free of R-HCQ, e.g., containing by weight 2% or less R-HCQ (e.g., 1% or less and 0.5% or less).

An exemplary pharmaceutical composition contains 50% to 70% of S-HCQ, and 30% to 50% of one or more of pharmaceutical excipients.

To carry out the method of this invention, a subject suffering from APS is typically administered with an effective amount of the pharmaceutical composition corresponding to a daily dose of 100 mg to 800 mg (e.g., 200 mg and 400 mg) of S-HCQ.

Administration of S-HCQ has fewer side effects, particularly respecting cardiotoxicity, as compared to R-HCQ and to racemic HCQ (i.e., an equimolar mixture of S-HCQ and R-HCQ).

In addition to S-HCQ, the pharmaceutical composition also includes a pharmaceutically acceptable excipient, which can be any physiologically inert excipient used in the pharmaceutical art, including but not limited to a binder, a diluent, a surfactant, a disintegrant, a lubricant, a glidant, and a coloring agent. For examples of an excipient, see US Patent Application Publication 2008/020634.

The pharmaceutical composition is provided in any form, e.g., granules, a tablet, a capsule, a pill, a powder, a solution, a suspension, and a syrup. It can be prepared following conventional methods described in many publications, see, e.g., US Patent Application Publication 2018/0194719.

The method of this invention is found surprisingly efficient in treating a subject suffering from primary APS (thrombotic APS and obstetric APS), secondary APS, and catastrophic APS.

Primary APS is a thrombophilic state in the absence of any comorbidity characterized by recurrent arterial and venous thrombosis, recurrent pregnancy loss, and the presence of circulating aPL antibodies responsible for thrombophilia and pregnancy morbidity. In a patient having secondary APS, there is a pre-existing autoimmune condition. Catastrophic APS, the most severe form of APS, is a multisystem autoimmune condition associated with aPL antibodies and characterized by vascular thromboses or pregnancy losses when there is simultaneous multi-organ failure with small vessel occlusion.

Varying from patient to patient, APS symptoms include signs such as blood clots, miscarriage, rash, chronic headaches, dementia, seizures, arterial thrombosis, autoimmune thrombocytopenia, autosomal dominant inheritance, blurred vision, central retinal artery occlusion, iritis, keratitis, lupus anticoagulant, retinal detachment, retinal vasculitis, scleritis, venous thrombosis, visual loss, and vitritis.

Not to be bound by theory, it is believed that HCQ treats APS by binding to β2-glycoprotein I (“β2-GP1”), a blood protein associated with APS. β2-GP1 circulates in blood at a high concentration, i.e., 0.2 mg/mL, capable of regulating blood coagulation. See McDonnell et al.

β2-GP1 exists in two conformations, i.e., a closed circular form and an open linear form. It is unclear what triggers β2-GP1 changing its conformation between these two forms. Among them, 90% of β2-GP1 moves in blood in the circular form. See Agar et al., Blood 116, 1336-43 (2010). In its linear form, β2-GP1 exposes two domains, i.e., an N-terminal Domain I (“DI”) and a C-terminal Domain V (“DV”). DI is a major region to receive an antibody, e.g., an aPL antibody. DV is responsible for binding to a blood cell membrane. When β2-GP1 changes from the circular form to the linear form, it promotes antibody binding to a blood cell membrane, thus initiating clotting reactions. This is achieved by forming a β2-GP1-antibody complex, a key pathogenic pathway to APS.

It is recognized that the β2-GP1-antibody complex exerts a coagulant effect by disrupting the anticoagulant annexin V shield on a blood cell, among other mechanisms. See McDonnell et al.

Annexin V, a cellular protein, inhibits formation of blood clots by binding to a phospholipid of a blood cell membrane, forming a shield that blocks an antibody from attacking the phospholipid. In an APS patient, the annexin V shield is disrupted by an antibody through β2-GP1.

HCQ is believed to break the pathogenic pathway described above by preventing β2-GP1 from changing its conformation to a linear form.

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever.

### Examples

### Example 1: Molecular Docking of S-HCQ and R-HCQ on β2-Glycoprotein I

Molecular docking was simulated to assess the binding stability of S-HCQ and R-HCQ with β2-GP1, the structure of which was obtained from the Protein Data Bank (PDB: 1AV1), a worldwide open-access archive of structural data of biological macromolecules. BIOVIA® Discovery Studio software (Dassault Systemes, San Diego, California) was used to align and edit protein structures and amino acid sequences. SwissDock, a docking service provided by Swiss Institute of Bioinformatics, performed computer analysis to predict hydrophobic regions of β2-GP1. UCSF Chimeram (University of California, San Francisco) software was used to visualize and analyze molecular structures.

Molecular docking helps understand how HCQ binds to β2-GP1 and inhibit its conformational changes. S-HCQ and R-HCQ were separately subjected to molecular docking simulation with β2-GP1. When binding to an antibody, β2-GP1 undergoes a conformational change from a closed form (circular) to an open form (linear). The latter promotes formation of β2-GP1-antibody complexes, causing thrombosis. Therefore, it is crucial to know whether the binding of HCQ can interfere with the conformational change of β2-GP1.

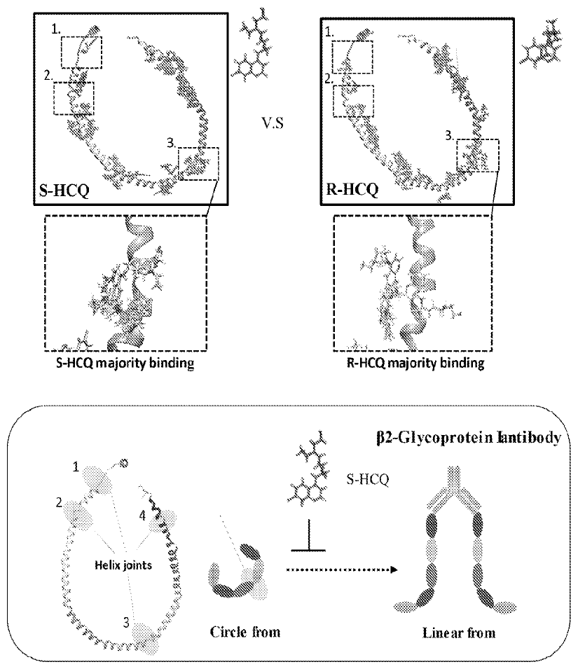

Molecular docking results are shown in FIG. 1. As illustrated, β2-GP1 contains a pseudocontinuous, amphipathic a-helix that has five domains and four joints (shown as 1, 2, 3, and 4 in the figure). Two adjacent a-helix domains are connected with a joint. The four joints, together or separately, enable movement of the a-helix thus changing β2-GP1 configuration between a circular form and a linear form.

Molecular docking indicated that a HCQ molecular can bind to any one of the four joints (1, 2, 3 and 4). Among them, binding to joint 3 is effective in inhibiting conformational changes. The strength of the binding is measured by affinity energies calculated in the molecular docking study. A higher affinity energy indicates a stronger binding.

S-HCQ binds to joint 3 at an affinity energy of 12.6 kcal/mol, a very high value. By fixing joint 3 of the β2-GP1 a-helix, S-HCQ inhibits its conformational change from a circular form to a linear form, which is required for antibody coupling with β2-GP1 to form a complex, a necessary APS pathogenic pathway. Thus, S-HCQ blocks this pathway, thereby effectively treating APS.

By contrast, R-HCQ binds to joint 3 at an affinity energy of only 10.5 kcal/mol, an energy level not very effective to prevent a conformational change in the β2-GP1 a-helix. Further, the binding angel of R-HCQ onto the β2-GP1 a-helix is different from that of S-HCQ, making R-HCQ less effective in suppressing configurational changes.

### Example 2: Inhibition of β2-GP1-Antibody Formation of by HCQ

In an in vitro study, S-HCQ and R-HCQ were tested to show their effectiveness in reducing binding of an aPL antibody and β2-GP1 using THP-1, a human monocyte cell line derived from a patient with monocytic leukemia.

THP-1 is a human peripheral blood monocyte that highly expresses β2-GP1 and is associated with increased APS in patients. Before treating with HCQ, THP-1 cells were fixed in a 3.7% paraformaldehyde solution, blocked in a phosphate-buffered saline (PBS) containing 1% bovine serum albumin (BSA), and incubated with a mouse aPL antibody (A500-006A, Bethyl Laboratories, Montgomery, Texas). Cellular immunofluorescent staining was used to determine β2-GP1 expression.

Separate samples were prepared for a dot blot assay as follows: a THP-1 total lysate was loaded to a polyvinylidene difluoride membrane, blocked with PBS containing 1% BSA, and incubated with mouse anti-β2-GP1 A500-006A, together with S-HCQ, R-HCQ, or racemic HCQ, each at a concentration of 10 mg/mL. A control sample was obtained following the same procedure described above except that no HCQ was added.

Subsequently, an ELISA assay was performed to determine whether S-HCQ or R-HCQ inhibiting binding of β2-GP1-antibody complexes to the THP-1 membrane. THP-1 cells thus treated were resuspended to a density of 3.6×10⁵ cells/mL in a medium containing 80% RPMI-1640 (Thermo Fisher Scientific, Waltham, Massachusetts), 20% anti-β2-GP1 immunoglobulin G (“IgG”) (0.2 mg/mL) in HEPES-buffered saline (HBS, pH 7.45), and S-HCQ. Three samples were each prepared from a medium having a different S-HCQ concentration, i.e., 1 μg/mL, 2.5 μg/mL, or 5 μg/mL. A control sample was obtained following the same procedure described above except that no S-HCQ was added. Optical absorbance was measured at 450 nm.

All assays above were performed in triplicate. Reported results are expressed as means±S.E.M. All statistical analyses were performed using a software under the trademark of GraphPad Prism® (Version 8.0. GraphPad Software Inc, San Diego, California). For comparisons between two groups, student's test was used. A p-value<0.05 was considered statistically significant.

Using cellular immunofluorescence staining, THP-1 cells in this study were found to express at a high level β2-GP1, confirmed as green round dots overlapping THP-1 cells, which were shown as round single-cell morphology with nucleic blue dots.

Dot blot analysis showed that S-HCQ inhibiting β2-GP1 and antibody binding at a surprising level of 97% (±2%, p<0.05). As a comparison, R-HCQ inhibited the β2-GP1 and antibody binding at only 23% (±17%), and racemic HCQ inhibited the binding at 80% (±4%).

The ELISA assay demonstrated that S-HCQ inhibited the β2-GP1 and antibody binding in a dose-dependent manner, i.e., an inhibition rate of 25% at 1 μg/mL S-HCQ, 60% at 2.5 μg/mL S-HCQ, and 90% at 5 μg/mL S-HCQ (p<0.05).

S-HCQ was found effectively inhibiting the β2-GP1 and antibody binding, surprisingly more effective than R-HCQ and racemic HCQ.

### Example 3: Restoration of Annexin A5 Anticoagulant Shield on a Cell Membrane by HCQ

As pointed out above, annexin A5, an endogenous protein, forms a shield on a blood cell surface inhibiting blood coagulation, thus reducing the risk of APS. In this example, S-HCQ significantly restored the annexin A5 anticoagulant shield, which was disrupted by an aPL antibody.

THP-1 cells were first maintained in a RPMI1640 medium (Thermo Fisher Scientific, Waltham, Massachusetts) containing 10% fetal bovine serum, 2 mM L-glutamine, and 50 U/mL penicillin-streptomycin antibiotics. They were then seeded at a density of 8×10⁴ cells/well in a 96-well culture plate and were allowed to reach confluence. Subsequently, THP-1 cells were treated with anti-β2-GP1 IgG in the presence of 0.5 μg/mL HCQ. Levels of annexin V on cell surfaces were determined by optical absorbance after the IgG-treated THP-1 cells were rinsed with HBS-CaCl₂) solution to remove free annexin V, leaving only annexin V that is attached to the cell surface.

Three samples were prepared each with one of three HCQ (i.e., S-HCQ, R-HCQ, and racemic HCQ) solutions. A comparative sample was obtained following the same procedure described above except that HCQ was not added. A patient serum containing β2-GP1 antibody was used as a control sample.

Cellular immunofluorescence staining confirmed a highly expression level of annexin V as green dots in immunofluorescent images. In addition, the expression of Annexin V in THP-1 was verified by western blot.

S-HCQ treated THP-1 cells had annexin V at a relative level of 3.45, as compared to a relative level of 2.54 found in R-HCQ treated THP-1 cells and a relative level of 3.09 found in racemic HCQ-treated THP-1 cells. The comparative sample showed only a relative level of 1 of annexin V.

The above results indicate that S-HCQ is surprisingly more potent than R-HCQ and racemic HCQ in treating APS.

### Example 4: In Vivo Clot Formation Reduction by HCQ

The therapeutic effect of HCQ on inhibiting the thrombosis was assessed by an APS-related thrombosis animal model.

### Isolation of Anti-β2-Glycoprotein I

Serum and plasma from 6 APS patients (i.e., Patients 1-6) were selected to participate in the study. Anticardiolipin (aCL), anti-β2GPI, and lupus anticoagulant (LA) activities were measured for APS confirmation. APS-derived anti-β2GPI samples were obtained by purification of serum samples using rProtein A/Protein G GraviTrap™ (Cytiva™, Merck KGaA, Darmstadt, Germany). The concentration of anti-β2GPI antibody in each sample was tested using binding to β2GPI by enzyme-linked immunosorbent assay (ELISA) (Eagle Biosciences, Inc., Amherst, New Hampshire).

In vitro endothelial cells activation was induced by anti-β2GPI antibodies obtained above. Endothelial cells (i.e., HUVECs) were seeded and incubated with an APS-derived anti-β2GPI antibody. As a positive control, some HUVECs were treated with lipopolysaccharide (LPS, 3 mg/mL). The surface expression of E-selectin, intercellular adhesion, and vascular cell adhesion molecule 1 (VCAM-1) were detected and found to have a positive correlation with the amount of β2GPI IgG.

### APS-Related Thrombosis Mice Model

C57BL/6 male mice at ages of 8-12 weeks (purchased from BioLasco, Taiwan) were used in this study. All procedures were approved by the Institutional Animal Care Committee of Taipei Medical University.

Venous thrombosis was induced in mice using an endothelial injury model. Mice were intravenously (IV) injected 7.5% FeCl₃ (positive control), saline (negative control), or an APS-derived anti-β2GP1 antibody (from Patient 2) at a dosage of 100, 200, or 300 AU (test group). They were anesthetized 72 hours after the injection. The right femoral vein was exposed and pinched with a pressure of 1500 g/mm² to induce formation of a thrombus.

It was determined that the anti-β2GP1 antibody effectively induced a thrombus at all three concentrations, i.e., 100, 200, and 300 AU. As such, the anti-β2GP1 antibody was injected to a mouse at 100 AU for all studies below.

### Clot Formation Reduced by Racemic-HCQ in APS-Related Thrombosis Mice Model

Following the procedure described above, a mouse (i.e., HCQ-treated mouse) was injected with 100 AU of the anti-β2GP1 antibody from Patient 5 and 2000 μg racemic-HCQ (200 μl at 10 mg/ml). As a control, a group of mice (n=4; APS-induced mice) were injected with only 100 AU of the anti-β2GP1 antibody. After 72 hours, the right femoral vein of each mouse was exposed and pinched with a pressure of 1500 g/mm² to induce formation of a thrombus. Thrombosis time (in minute) was recorded. A group of healthy mice (n=2) showed a thrombosis time of 5 minutes, while the APS-induced mice showed an average thrombosis time of 2 minutes. By contrast, the HCQ treated mouse had a thrombosis time of 5 minutes, the same as the healthy mice. The results indicated that the anti-β2GP1 antibody accelerated thrombosis in APS-induced mice and, on the other hand, racemic-HCQ inhibited the function of the anti-β2GP1 antibody.

Clots were removed from the femoral vein after pinch for 5 minutes. Racemic-HCQ significantly reduced the thrombus size as compared to the mice group without HCQ treatment.

The results indicates that racemic-HCQ, including both S-HCQ and R-HCQ, is useful in treating APS by reducing clog formation in APS patients.

### Clot Formation Reduction by S-HCQ in APS-Related Thrombosis Mice Model

Risk factors for APS-related thrombosis and venous thromboembolism (VTE) overlap and are mostly associated with endothelial dysfunction (ED). E-selectin and VCAM-1 are associated with a high risk of APS-related thrombosis.

Based on the APS animal model described above, R-HCQ and S-HCQ were administered to mice for reducing thrombosis. Mice were divided into 6 groups, each of which was injected with (1) IgG as a control group, (2) 100 AU of the anti-β2GPI antibody as a comparative group, (3) a combination of 100 AU of the anti-B2GPI antibody and 300 μg of S-HCQ as treatment group 3, (4) a combination of 100 AU of the anti-B2GPI antibody and 200 μg of S-HCQ as treatment group 4, (5) a combination of 100 AU of the anti-B2GPI antibody and 200 μg of R-HCQ as treatment group 5, or (6) a combination of 100 AU of the anti-β2GP1 antibody and 100 μg of R-HCQ as treatment group 6.

Serum expression levels of E-selectin and VCAM-1, two biomarkers of APS-related thrombosis, were measured. Percentages of inhibition in treatment groups 3-6 were calculated based on the expression levels of E-selectin and VCAM-1 relative to the comparative group. See Table 1 below for the results. A low expression level of E-selectin or VCAM-1 indicates high inhibition of thrombosis.

As shown in Table 1, mice treated with 300 μg or 200 μg of S-HCQ had much lower expressions of both E-selectin and VCAM-1 as compared to mice treated with R-HCQ. It was surprisingly found that mice treated with 200 μg of S-HCQ inhibited thrombosis by 51.4% as compared to mice treated with 200 μg of R-HCQ by only 26.5%.

TABLE 1

| HCQ | Dose | Biomarker | Thrombosis | Average | Inhibition | Average |
|---|---|---|---|---|---|---|
| S- | 300 μg | E- | 40.6% | 44.2% | 59.4% | 55.7% |
| HCQ | | seclection | | | | |
| | | VCAM-1 | 47.8% | | 52% | |
| | 200 μg | E-seclectin | 46.2% | 48.55% | 53.8% | 51.4% |
| | | VCAM-1 | 50.9% | | 49% | |
| R- | 200 μg | E-seclectin | 70% | 72.5% | 30% | 26.5% |
| HCQ | | VCAM-1 | 75% | | 23% | |
| | 100 μg | E-seclectin | 74.8% | 79.25% | 25.2% | 20.75% |
| | | VAM-1 | 83.7% | | 16.3% | |

### Other Embodiments

All of the features disclosed in this specification may be combined in any combination. Each feature disclosed in this specification may be replaced by an alternative feature serving the same, equivalent, or similar purpose. Unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method of treating antiphospholipid syndrome (APS), the method comprising:
identify a subject suffering from APS, and
administering to the subject an effective amount of a pharmaceutical composition containing (S)-(±)-hydroxychloroquine and a pharmaceutically acceptable excipient, thereby treating APS, wherein the pharmaceutical composition is substantially free of (R)-(−)-hydroxychloroquine.

2. The method of claim 1, wherein the S-hydroxychloroquine is in the form of a pharmaceutically acceptable salt.

3. The method of claim 2, wherein the pharmaceutically acceptable salt is a hydrochloride salt, a sulfate salt, or a phosphate salt.

4. The method of claim 1, wherein a dose of the S-hydroxychloroquine administered to the subject is 100 mg to 800 mg per day.

5. The method of claim 4, wherein the dose is 150 mg to 500 mg per day.

6. The method of claim 1, wherein the pharmaceutical composition is in the form of granules, a tablet, a capsule, a pill, a powder, a solution, a suspension, or a syrup.

7. The method of claim 1, wherein the APS is primary APS, secondary APS, or catastrophic APS.

8. The method of claim 1, wherein the APS is a thrombotic APS or an obstetric APS.
